(19) United States
(12) Patent Application Publication
(10) Pub. No.: US 2025/0084035 A1
(43) Pub. Date: Mar. 13, 2025

(54) POLYMORPHIC FORM OF MEISOINDIGO AND MODIFIED FORMULATION OF MEISOINDIGO

(71) Applicant: STATIA BIOPHARMA INC., Flushing, NY (US)

(72) Inventor: Longgui Wang, Flushing, NY (US)

(21) Appl. No.: 18/955,955

(22) Filed: Nov. 21, 2024

Related U.S. Application Data
(63) Continuation of application No. 17/298,528, filed on May 28, 2021.
(60) Provisional application No. 62/776,965, filed on Dec. 7, 2018.

(51) Int. Cl. C07D 209/18

(52) U.S. Cl. CPC C07D 209/18; C07B 2200/13

(57) ABSTRACT

The present invention relates to a novel crystal form, manufacturing procedures, pharmaceutical compositions, formulations and medicaments comprising a N-methylisoindigo crystalline, methods of preparation, and the use of the N-methylisoindigo crystalline to prepare a medicament to prevent cancer, or treat cancer or an inflammatory-related disease associated with pro-inflammatory cytokine expression and/or reduced expression of anti-inflammatory cytokines.

### CROSS-REFERENCE TO RELATED APPLICATIONS

The present application is a Continuation of U.S. patent application Ser. No. 17/298,528, filed on May 28, 2021, which is the U.S. National Stage of International Patent Application No. PCT/US2019/062704, filed on Nov. 21, 2019, which claims priority to U.S. Provisional Patent Application No. 62/776,965 filed Dec. 7, 2018, which is hereby incorporated by reference in its entirety.

### TECHNICAL FIELD

The present invention relates to pharmaceutical compositions, more particularly a novel crystalline form of meisoindigo, methods of preparation, and methods of preventing cancer, treating cancer, or treating inflammatory-related diseases associated with inhibiting cyclin-dependent kinases, pro-inflammatory cytokine expression, or reduced expression of anti-inflammatory cytokines.

### BACKGROUND OF THE INVENTION

Prevention and treatment of cancer have significantly improved in the United States during the past decade because of advancements in epidemiology, the technology of treatment, and the ability to deliver an earlier diagnosis. Finding a cure for a diversity of cancers, such as lung, breast, prostate, colon, and others, however, is still a major challenge. Current approaches for the treatment of cancers are, however, still limited to the lengthening of life, or the increase in the quality of life. Additionally, most meaningful therapeutics still have significant side effects. Therefore, it is imperative to find more effective therapeutic agents with lower side effects.

Tumor cells are characterized by uncontrolled cell proliferation due to the loss of the integration and coordination of extracellular signals with the cell cycle machinery. A typical cell cycle is classified into G1, S, G2 and M phases. In mammalian cells, proliferation is controlled in the G1 phase of the cell cycle. At the restriction point, cells can have different destinies. Examples of these cell destinies include: 1) leaving the cell cycle and entering a reversible quiescence phase; 2) exiting cell cycle and undergoing apoptosis; 3) differentiating and irreversibly exiting from the cell cycle; and 4) passing through the restriction point and becoming largely independent of extracellular signals and progress automatically through subsequent cell cycle phases (S, G2, M) to the next G1 phase. A variety of proteins are in turn responsible for the regulated progression of cells through the cell cycle. The key components of cell cycle machinery are the cyclins, the cyclin-dependent kinases (CDKs) and their inhibitors. Cyclins are a remarkably diverse family of proteins, which are synthesized from the mid/late of G1 phase until the M phase of the cell cycle and then rapidly degraded. A CDK typically contains a catalytic domain of 300 amino acids, which is inactive by itself. Cdks become active by binding to a cyclin. The activity of cdks is inhibited by their endogenous inhibitors (CDK inhibitors, or cdkIs include p15/p16/p18/p19 and p21/p27). Specific cyclin/CDK complexes are formed at specific stages of the cell cycle, and their activities are required for progression of the cell cycle through S phase and mitosis.

Over-activation of CDKs is a character of a majority of human tumor cells. Strategies have been developed to modulate CDK activity for therapeutic intervention by either directly targeting the catalytic CDK Subunit or indirectly affecting the CDK regulatory pathways 3). Small molecule CDK inhibitors were designed to interact specifically with the ATP binding site of CDKs, such as flavopiridol congeners, poly sulfates, toyocamycin derivatives, etc. Anticancer effects have been shown in clinical trials for those agents. Modulation of CDK activities can be achieved by regulating the phosphorylation of CDKS or altering the expression of the CDKs or their inhibitors (CDKIs). It is difficult to find specific modulators that do not interfere with other cell cycle components and do not affect normal cells.

Our previous studies demonstrated that meisoindigo, a second generation of indirubins, arrests leukemia cells at G phase, inhibits expression of oncogene c-myb, and induces cell differentiation and maturation at low concentrations (low toxicity) in which cell growth is completely inhibited without a decrease in cell viability.

There is a need for a modified meisoindigo formulation that has even better solubility and bioavailability and/or longer shelf-life.

### SUMMARY OF THE INVENTION

The present invention provides a novel crystal form (solid form) of Meisoindigo (Crystal Form I), which exhibits excellent therapeutic efficacy against various autoimmune diseases/inflammatory diseases as well as cancers. The present invention further provides a process for preparing the crystal form (Form 1) as well as pharmaceutical compositions and dosages comprising the novel crystal form.

In some embodiments, the present invention provides a solid form of N-methylisoindigo or a solid crystal form of N-methylisoindigo (Crystal Form I) having an X-ray powder diffraction pattern comprising a peak, in terms of 2-theta, at about 7.71°.

In one embodiment, the solid form or solid crystal form has an X-ray powder diffraction pattern comprising peaks, in terms of 2-theta, at about 7.71°, about 17°, about 18°, and about 29°.

In another embodiment, the solid form or solid crystal form has an X-ray powder diffraction pattern substantially as shown in FIG. 5.

In certain aspects, the solid form or solid crystal form has an infrared spectrum of N-methylisoindigo substantially as shown in FIG. 1. In other aspects, the solid form or solid crystal form has an NMR spectrum of N-methylisoindigo substantially as shown in FIG. 2.

In yet other aspects, the solid form or solid crystal form has a differential scanning calorimetry (DSC) thermogram comprising an endothermic peak at about 235° C. to 237° C. In one aspect, the solid form or solid crystal form has a differential scanning calorimetry (DSC) thermogram substantially as shown in Table 1.

In some embodiments, the solid form or solid crystal form has a particle size distribution with an average particle size d50 below 25 μm. In certain embodiments, the solid form or solid crystal form has a particle size distribution with an average particle size d50 below 25 μm, below 24 μm, below 23 μm, below 22 μm, below 21 μm, below 20 μm, below 19 μm, below 18 μm, below 17 μm, or below 16 μm. In other embodiments, the solid form or solid crystal form has a particle size distribution with an average particle size d50 of between 1 μum and 25 μm (e.g., between 1 μm and 25 μm, between 1 μm and 20 μm, between 5 μm and 25 μm, between 5 μm and 20 μm, between 10 μm and 25 μm, or between 10 μm and 20 μm).

In certain aspects, the solid form or solid crystal form has a particle size distribution ratio (d90-d10)/d50 of less than 2.50. In some aspects, the particle size distribution ratio (d90-d10)/d50 is less than 2.50, less than 2.40, less than 2.30, less than 2.20, less than 2.10, less than 2.00, or less than 1.90.

In other aspects, the solid form or solid crystal form has a maximum particle size below 100 μm. In certain aspects, the maximum particle size is below 100 μm, below 90 μm, below 80 μm, below 70 μm, below 60 μm, or below 50 μm.

In certain aspects, the particle size distribution facilitates pharmaceutical processing during formulation of the solid form or solid crystal form. In other aspects, the particle size distribution enhances the stability and bioavailability of the solid form or solid crystal form.

In some embodiments, the present invention provides a pharmaceutical composition comprising a solid form or solid crystal form disclosed herein, and a pharmaceutically acceptable carrier.

In certain embodiments, the solid form or solid crystal form is present in the pharmaceutical composition in an amount of at least about 90% by weight. In other embodiments, the solid form or solid crystal form is present in the pharmaceutical composition in an amount of at least about 10% by weight, at least about 20% by weight, at least about 30% by weight, at least about 40% by weight, at least about 50% by weight, at least about 60% by weight, at least about 70% by weight, at least about 80% by weight, or at least about 90% by weight.

In yet other embodiments, the solid form or solid crystal form is present in the pharmaceutical composition in an amount of between 1% by weight and 90% by weight (i.e., between 1% by weight and 90% by weight, between 1% by weight and 80% by weight, between 1% by weight and 70% by weight, between 1% by weight and 60% by weight, between 1% by weight and 50% by weight, between 1% by weight and 40% by weight, between 1% by weight and 30% by weight, between 1% by weight and 20% by weight, or between 1% by weight and 10% by weight).

In certain aspects, the solid form or solid crystal form is substantially purified. In other aspects, solid form or solid crystal form is crystalline.

In certain embodiments, the present invention provides a process for preparing a solid form or solid crystal form disclosed herein, the process comprising precipitating a crystalline form from a solution comprising an organic solvent. In one embodiment, the solution comprises glacial acetic acid. In another embodiment, the solution further comprises N-methylisatin, oxindole, and/or HCl.

In other embodiments, the present invention provides a solid form or a solid crystal form of N-methylisoindigo prepared by a process disclosed herein and recrystallized in any other acceptable organic solvent, and preferably in glacial acetic acid.

In some embodiments, the present invention provides a method of treating cancer, comprising administering a solid form or solid crystal form of N-methylisoindigo disclosed herein or a pharmaceutical composition disclosed herein to a patient in need thereof.

In other embodiments, the present invention provides a method of treating an inflammatory-related disease associated with cytokine expression levels, comprising administering a solid form or solid crystal form of N-methylisoindigo disclosed herein or a pharmaceutical composition disclosed herein to a patient in need thereof.

In certain aspects, the present invention provides a pharmaceutical composition consisting essentially of a solid form or solid crystal form of N-methylisoindigo disclosed herein.

### BRIEF DESCRIPTION OF THE DRAWINGS

FIG. 1 is an infrared spectrum of N-methylisoindigo.

FIG. 2 is an NMR spectrum of N-methylisoindigo.

FIG. 3 is a mass spectrum of N-methylisoindigo (positive Q1 scan).

FIG. 4 is a mass spectrum of N-methylisoindigo (negative Q1 scan).

FIG. 5 shows a comparison of the X-ray powder diffraction patterns. Unsmoothed XRD data for the four N-methylisoindigo samples. The peak of ˜40,000 counts at 7.71 deg is truncated so that a detailed view of the data in the range 5<2Theta<55 deg is possible. Note the extra peaks in sample 0501 (blue curve) that is further detailed in FIG. 6 and Table 2.

FIG. 6 plots results of a peak selection analysis. Detail of the 5<2Theta<31 deg region of all 4 XRD patterns of all 4 N-methylisoindigo samples. The extra peaks in sample 0501 are labeled a through m and are tabulated in Table 2. The maximum intensity is observed at about 7.71 deg (d-space=11.5 Å) and is about 40,000 counts for all samples, and is consistent with the notion that the molecular plane of N-methylisoindigo is nearly parallel to this axis in the unit cell of the crystal structure.

FIG. 7 depicts a calculated powder diffraction pattern (Cu Kα radiation, λ=1.5418Å) of isoindigo, based upon the coordinates of the single crystal, Cambridge Structure Database reference code “ISOIND.”

FIG. 8 is an example of ¹H-NMR spectrum of samples 1 to 6 N-methylisoindigo.

FIG. 9 is an example of ¹³C-NMR spectrum of samples 1 to 6 N-methylisoindigo.

FIG. 10A shows a comparison of the X-ray powder diffraction patterns for samples 1 to 6. Individual X-ray powder diffractions patterns for sample 1, 2, 3, 4, 5, and 6 are presented in FIGS. 10B, 10C, 10D, 10E, 10F, and 10G, respectively.

FIG. 11 shows examples of particle sizes and their distribution of Crystal Form I of meisoindigo.

### DETAILED DESCRIPTION OF THE INVENTION

Herein, the inventors disclose a novel and unique crystal form (Crystal Form I) and a method of manufacturing crystalline N-methylisoindigo that has shown excellent efficacy in treating various autoimmune/inflammatory diseases and cancers.

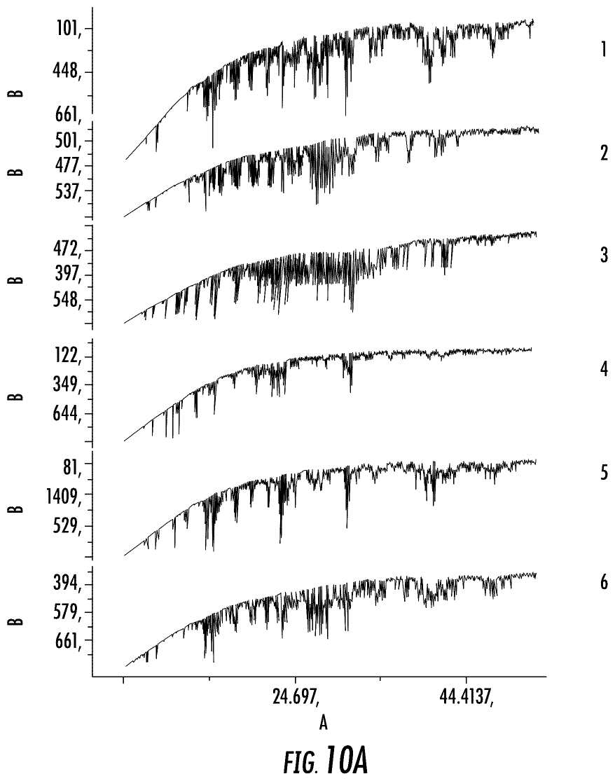

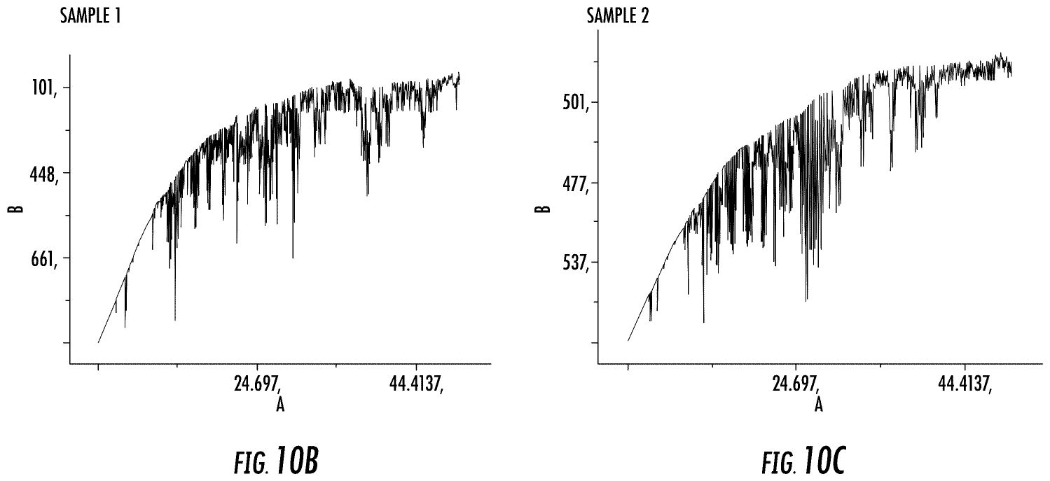

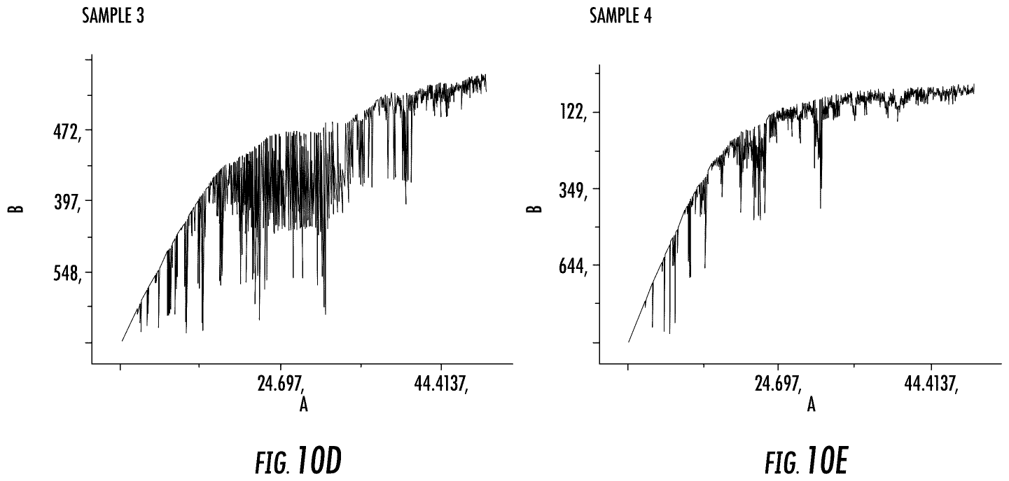

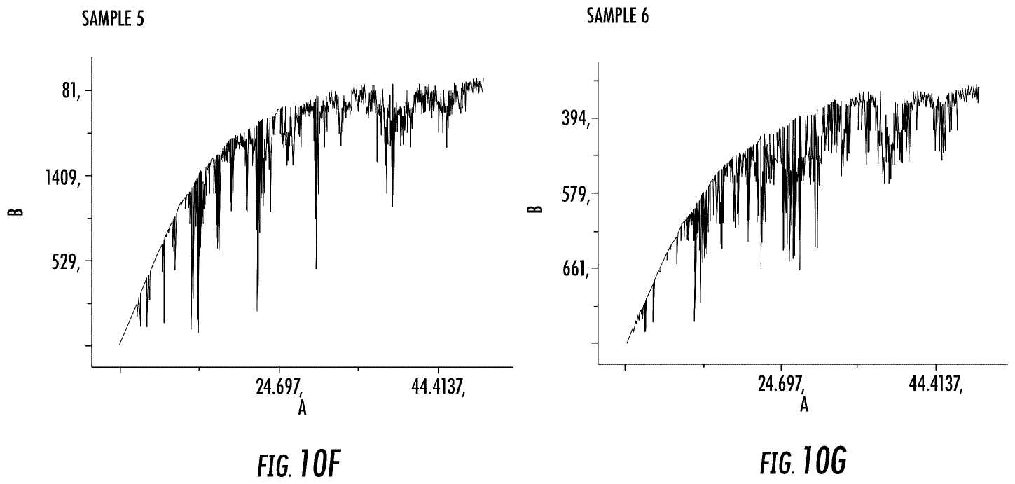

In certain aspects, the present invention provides a solid form of N-methylisoindigo or a solid crystal form of N-methylisoindigo having an X-ray powder diffraction pattern substantially as shown in FIG. 10A. In one aspect, the solid form of N-methylisoindigo or solid crystal form of N-methylisoindigo has an X-ray powder diffraction pattern substantially as shown in FIG. 10B. In another aspect, the solid form of N-methylisoindigo or solid crystal form of N-methylisoindigo has an X-ray powder diffraction pattern substantially as shown in FIG. 10C. In another aspect, the solid form of N-methylisoindigo or solid crystal form of N-methylisoindigo has an X-ray powder diffraction pattern substantially as shown in FIG. 10D. In another aspect, the solid form of N-methylisoindigo or solid crystal form of N-methylisoindigo has an X-ray powder diffraction pattern substantially as shown in FIG. 10E. In another aspect, the solid form of N-methylisoindigo or solid crystal form of N-methylisoindigo has an X-ray powder diffraction pattern substantially as shown in FIG. 10F. In another aspect, the solid form of N-methylisoindigo or solid crystal form of N-methylisoindigo has an X-ray powder diffraction pattern substantially as shown in FIG. 10G.

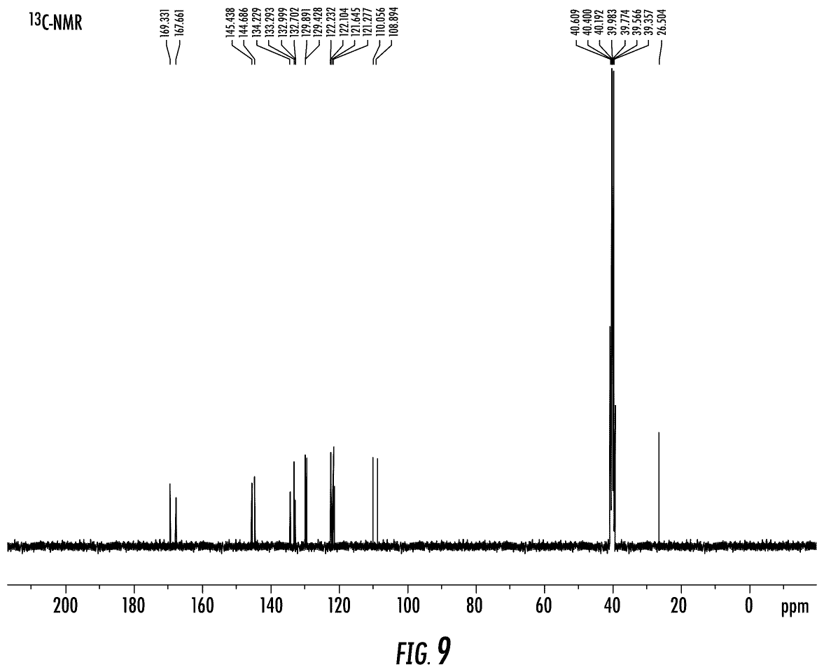

In other aspects, the solid form or solid crystal form has an NMR spectrum of N-methylisoindigo substantially as shown in FIG. 8 and/or FIG. 9.

In certain embodiments, the solid form or solid crystal form is in the form of a pharmaceutical composition or formulation which is ready for use to be administered to a patient or patient in need thereof. The solid form or solid crystal form may be administered by the oral, intravenous, topical, local installations, intraperitoneal or nasal route. Said compositions can be utilized to achieve the desired pharmacological effect by administration to a patient in need thereof.

A patient, for the purpose of this invention, is a mammal, including a human, in need of treatment for the particular condition or disease. Therefore, the present invention includes pharmaceutical compositions that are comprised of a pharmaceutically acceptable carrier and a pharmaceutically effective amount of the solid form or solid crystal form. A pharmaceutically acceptable carrier is preferably a carrier that is relatively nontoxic and innocuous to a patient at concentrations consistent with effective activity of the active ingredient so that any side effects ascribable to the carrier do not vitiate the beneficial effects of the solid form or solid crystal form.

A pharmaceutically effective amount of a solid form or solid crystal form is preferably that amount which produces a result or exerts an influence on the particular condition being treated. The solid form or solid crystal form of the present invention can be administered with pharmaceutically-acceptable carriers well known in the art using any effective conventional dosage unit forms, including immediate, slow and timed release preparations, orally, parenterally, topically, nasally, ophthalmically, optically, sublingually, rectally, vaginally, and the like.

It is possible for the compounds according to the invention to have systemic and/or local activity. For this purpose, they can be administered in a suitable manner, such as, for example, via the oral, parenteral, pulmonary, nasal, sublingual, lingual, buccal, rectal, vaginal. dermal, transdermal, conjunctival, otic route or as an implant or stent.

For these administration routes, it is possible for the compounds according to the invention to be administered in suitable administration forms.

For oral administration, it is possible to formulate the compounds according to the invention to dosage forms known in the art that deliver the compounds of the invention rapidly and/or in a modified manner, such as, for example, tablets (uncoated or coated tablets, for example with enteric or controlled release coatings that dissolve with a delay or are insoluble), orally-disintegrating tablets, films/wafers, films/lyophylisates, capsules (for example hard or soft gelatin capsules), sugar-coated tablets, granules, pellets, powders, emulsions, suspensions. aerosols or solutions. It is possible to incorporate the compounds according to the invention in crystalline and/or amorphised and/or dissolved form into said dosage forms.

Parenteral administration can be effected with avoidance of an absorption step (for example intravenous, intraarterial, intracardial, intraspinal or intralumbal) or with inclusion of absorption (for example intramuscular, subcutaneous, intracutaneous, percutaneous or intraperitoneal). Administration forms which are suitable for parenteral administration are. inter alia, preparations for injection and infusion in the form of solutions, suspensions, emulsions, lyophylisates or sterile powders.

Examples which are suitable for other administration routes are pharmaceutical forms for inhalation [inter alia powder inhalers, nebulizers], nasal drops, nasal solutions, nasal sprays; tablets/films/wafers/capsules for lingual, sublingual or buccal administration; suppositories: eye drops, eye ointments, eye baths, ocular inserts, ear drops, car sprays, ear powders, ear-rinses, car tampons, vaginal capsules, aqueous suspensions (lotions, mixturae agitandae), lipophilic suspensions, emulsions, ointments, creams, transdermal therapeutic systems (such as, for example, patches), milk, pastes, foams, dusting powders, implants or stents.

The compounds according to the invention can be incorporated into the stated administration forms. This can be effected in a manner known per se by mixing with pharmaceutically suitable excipients. Pharmaceutically suitable excipients include, inter alia.
fillers and carriers (for example cellulose, microcrystalline cellulose (such as, for example, Avicel”), lactose, mannitol, starch, calcium phosphate (such as, for example, Di-Cafos*)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat),
solvents (for example water, ethanol, isopropanol, glycerol, propylene glycol, medium chain-length triglycerides fatty oils. liquid polyethylene glycols, paraffins),
surfactants, emulsifiers, dispersants or wetters (for example sodium dodecyl sulfate), lecithin, phospholipids, fatty alcohols (such as, for example, Lanette*), sorbitan fatty acid esters (such as, for example, Span”), polyoxyethylene sorbitan fatty acid esters (such as, for example, Tween4), polyoxyethylene fatty acid glycerides (such as, for example, Cremophor*). polyoxethylene fatty acid esters, polyoxyethylene fatty alcohol ethers, glycerol fatty acid esters, poloxamers (such as, for example, Pluronic*),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (for example highly-disperse silicas),
viscosity-increasing agents, gel formers, thickeners and/or binders (for example polyvinylpyrrolidone, methylcellulose, hydroxypropylmethylcellulose, hydroxypropyl-cellulose, carboxymethylcellulose-sodium, starch, carbomers, polyacrylic acids (such as, for example, Carbopol*); alginates, gelatine).
disintegrants (for example modified starch, carboxymethylcellulose-sodium, sodium starch glycolate (such as, for example, Explotab*), cross-linked polyvinylpyrrolidone, croscarmellose-sodium (such as, for example, AcDiSof)),
flow regulators, lubricants, glidants and mould release agents (for example magnesium stearate, stearic acid, talc, highly-disperse silicas (such as, for example, Aerosil*)),
coating materials (for example sugar, shellac) and film formers for films or diffusion membranes which dissolve rapidly or in a modified manner (for example polyvinylpyrrolidones (such as, for example, Kollidon*), polyvinyl alcohol. hydroxypropylmethylcellulose, hydroxypropylcellulose, ethylcellulose, hydroxypropyl-methylcellulose phthalate, cellulose acetate, cellulose acetate phthalate, polyacrylates, polymethacrylates such as, for example, Eudragit*)), capsule materials (for example gelatine, hydroxypropylmethylcellulose),
synthetic polymers (for example polylactides, polyglycolides, polyacrylates, polymethacrylates (such as, for example, Eudragit*), polyvinylpyrrolidones (such as, for example, Kollidon*), polyvinyl alcohols, polyvinyl acetates, polyethylene oxides, polyethylene glycols and their copolymers and blockcopolymers), plasticizers (for example polyethylene glycols, propylene glycol, glycerol, triacetine, triacetyl citrate, dibutyl phthalate), penetration enhancers,
stabilisers (for example antioxidants such as, for example, ascorbic acid, ascorbyl palmitate, sodium ascorbate, butylhydroxyanisole, butylhydroxytoluene, propyl gal late), preservatives (for example parabens, sorbic acid, thiomersal, benzalkonium chloride, chlorhexidine acetate. sodium benzoate),
colorants (for example inorganic pigments such as, for example, iron oxides, titanium dioxide).
flavorings, sweeteners, flavor-and/or odor-masking agents,

The present invention furthermore relates to pharmaceutical compositions which comprise the crystalline forms of N-methylisoindigo disclosed herein, conventionally together with one or more pharmaceutically suitable excipient(s), and to their use according to the present invention.

Various diseases and disorders can be treated with the crystalline forms of N-methylisoindigo disclosed herein. These diseases and disorders are described in greater detail below.

### Irregular and/or Abnormal Inflammation

Irregular and/or abnormal inflammation is a major component of a wide range of human diseases. People suffering from multiple degenerative disorders often exhibit excess levels of pro-inflammatory markers in their blood. One type of such pro-inflammatory markers is pro-inflammatory mark cytokines including IL-1α, β, IL-2, IL-3, IL-6, IL-7, IL-9, IL-12, IL-17, IL-1, IL-23, TNF-α, LT, LIF, Oncostatin, and IFNc1α, β, γ.

A non-limiting list of common medical problems that are directly caused by inflammatory cytokines include: arthritis where inflammatory cytokines destroy lead to lesion in the synovial membrane and destruction of joint cartilage and bone; kidney failure where inflammatory cytokines restrict circulation and damage nephrons; lupus wherein inflammatory cytokines induce an autoimmune attack; asthma where inflammatory cytokines close the airway; psoriasis where inflammatory cytokines induce dermatitis; pancreatitis where inflammatory cytokines induce pancreatic cell injury; allergy where inflammatory cytokines induce autoimmune reactions; fibrosis where inflammatory cytokines attack traumatized tissue; surgical complications where inflammatory cytokines prevent healing; anemia where inflammatory cytokines attack erythropoietin production; and fibromyalgia where inflammatory cytokines are elevated in fibromyalgia patients. Other diseases associated with chronic inflammation include cancer, which is caused by chronic inflammation; heart attack where chronic inflammation contributes to coronary atherosclerosis; Alzheimer's disease where chronic inflammation destroys brain cells; congestive heart failure where chronic inflammation causes heart muscle wasting; stroke where chronic inflammation promotes thrombo-embolic events; and aortic valve Stenosis where chronic inflammation damages heart Valves. Arteriosclerosis, osteoporosis, Parkinson's disease, infection, inflammatory bowel disease including Crohn's disease and ulcerative colitis as well as multiple sclerosis (a typical autoimmune inflammatory-related disease) are also related to inflammation. Some diseases in advanced stages can be life-threatening. Several methodologies are available for the treatment of such inflammatory diseases; the results, however, are generally unsatisfactory as evidenced by a lack of efficacy and drug-related side effects associated therewith.

### Inflammatory Bowel Disease

Inflammatory bowel disease (IBD) comprises Crohn's disease (CD) and ulcerative colitis (UC), both of which are idiopathic chronic diseases occurring with an increasing frequency in many parts of the world. In the United States, more than 600,000 are affected every year. IBD can involve either or both small and large bowel. CD can involve any part of the gastrointestinal tract, but most frequently involves the distal small bowel and colon. It either spares the rectum or causes inflammation or infection with drainage around the rectum. UC usually causes ulcers in the lower part of the large intestine, often starting at the rectum. Symptoms vary but may include diarrhea, fever, and pain. Patients with prolonged UC are at an increased risk of developing colon cancer. There is currently no satisfactory treatment, as the cause for IBD remains unclear although infectious and immunologic mechanisms have been proposed. IBD treatments aim at controlling inflammatory symptoms, conventionally using cortical steroids, aminosalicylates and standard immunosuppressive agents such as azathioprine (6-mercaptopurine), methotrexate and cyclosporine. Of these, the only disease-modifying therapies are the immunosuppressive agents: azathioprine and methotrexate, both of which have a slow onset of action and only moderate efficacy. Long-term therapy may cause liver damage (fibrosis or cirrhosis) and bone marrow suppression. Also, patients often become refractory to such treatment. Other therapeutic regimes merely address symptoms.

### Psoriasis

Psoriasis is one of the most common immune-mediated chronic skin diseases that come in different forms and varied levels of severity, affecting approximately 2% or more than 4.5 million people in the United States of which 1.5 million are considered to have a moderate to severe form of the disease. Ten to thirty percent of patients with psoriasis also develop a form of arthritis—Psoriatic arthritis, which damages the bone and connective tissue around the joints. Psoriasis appears as patches of raised red skin covered by a flaky white buildup. It may also have a pimple-ish (pustular psoriasis) or burned (erythrodermic) appearance. Psoriasis may also cause intense itching and burning. Patients suffer psychologically as well as physically. Several modalities are currently available for treatment of psoriasis, including topical treatment, phototherapy, and systemic applications. However, they are generally considered to be only disease suppressive and disease-modifying. And none of them are curative. Moreover, many treatments are cither cosmetically undesirable, inconvenient for long-term use, or associated with significant toxicity.

With increased understanding of the biological properties of psoriasis over the past 2 decades, biologic therapies targeting the activity of T lymphocytes and cytokines responsible for the inflammatory nature of this disease have become available. Currently, drugs prescribed for psoriasis include those TNF-α inhibitors initially used for rheumatoid arthritis (RA) treatment, ENBREL® (etanercept), REMICADE® (infliximab) and HUMIRA® (adalimumab), and T-cell inhibitor AMEVIVE® (alefacept) from Biogen approved in 2002 and RAPTIVA® (efalizumab) from Genentech/Xoma approved in 2003. More recently, biologics, such as, antibodies against IL-17 (e.g., ixekizumab from Eli Lilly) or against IL-23 (e.g., guselkumab from Johnson & Johnson) have been developed. AMEVIVE® (alefacept) is an immunoglobulin fusion protein composed of the first extracellular domain of human LFA-3 fused to the hinge, C(H)2 and C(H)3 domains of human IgG(1). It inhibits T cell proliferation through NK cells. RAPTIVA® (efalizumab) is also known as anti-CD11a, a humanized monoclonal antibody which targets the T cell adhesion molecule, leukocyte function-associated antigen-1 (LFA-1). Prevention of LFA-1 binding to its ligand (ICAM-1, intercellular adhesion molecule-1) inhibits lymphocyte activation and migration, resulting in a decreased lymphocyte infiltration, thereby limiting the cascade of events eventually leading to the signs and symptoms of psoriasis. Potential side effects for the TNF-α inhibitor, however, are severe, including the development of lymphoma, worsening congestive heart failure, resulting in serious infection and sepsis, and exacerbations of multiple sclerosis and central nervous system problems. While side effects of the T-cell inhibitors of AMEVIVE® (alefacept) or RAPTIVA® (efalizumab) may be more tolerable in psoriasis treatment, these therapies act as immunosuppressive agents. Immunosuppressive agents have the potential to increase the risk of infection, reactivate latent, chronic infections or increase the risk of cancer development.

Although many advances have been made in the understanding of the biological properties of psoriasis over the past two decades and an unconventional treatment for psoriasis has become available as described above, much of the suffering it produces is still not adequately addressed. A Survey of over 40,000 American patients with psoriasis performed by the National Psoriasis Foundation in 1998 showed 79% of the younger patients felt frustrated by the ineffectiveness of their treatment. Of those with severe disease, 32% felt their treatment was not aggressive enough.

### Rheumatoid Arthritis

Rheumatoid arthritis (RA) represents another example of troublesome inflammatory disorders. It is a common chronic inflammatory-related disease characterized by chronic inflammation in the membrane lining (the synovium) of the joints and/or other internal organs. The inflammatory cells can also invade and damage bone and cartilage. The joint involved can lose its shape and alignment, resulting in loss of movement. Patients with RA have pain, stiffness, warmth, redness, and swelling in the joint, and other systemic symptoms like fever, fatigue, and anemia. Approximately 1% of the population or 2.1 million in the U.S. are currently affected, of which more are women (1.5 million) than men (0.6 million). The pathology of RA is not fully understood although the cascade of improper immunological reactions has been postulated as a mechanism. Conventional treatment is unfortunately inefficient in RA (29). The disease does not respond completely too symptomatic medications including corticosteroids and non-steroidal anti-inflammatory drugs (NSAIDs) used since the 1950s. Also, these medications carry a risk of serious adverse effects. The therapeutic effects of disease-modifying antirheumatic drugs (DMARDs) such as Methotrexate (MTX) are often inconsistent and short-lived.

A new class of biologic DMARDs (disease-modifying antirheumatic drugs) for the treatment of RA has recently been developed based on an understanding of the role of cytokines, TNF-α, and IL-1, in the inflammatory process. The FDA has approved several such DMARDs including ENBREL® (etanercept) from Immunex/Amgen Inc. in 1998, REMICADE® (infliximab) from Centocor/Johnson & Johnson, HUMIRA® (adalimumab) from Abbott

Laboratories Inc. in 2002, and KINERET® (anakinra) from Amgen in 2001. ENBREL® (etanercept) is a soluble TNF receptor (TNFR) recombinant protein. REMICADE® (infliximab) is a humanized mouse (chimeric) anti-TNF-α monoclonal antibody. HUMIRA® (adalimumab) is a fully human anti-TNF monoclonal antibody created using phage display technology resulting in an antibody with human derived heavy and light chain variable regions and human IgG1:k constant regions. All these 3 protein-based drugs target and bind to TNF-α to block the effects of TNF-α. KINERET® (anakinra) is a recombinant IL-1 receptor antagonist, which is similar to native human IL-1Ra, except for the addition of a single methionine residue at its amino terminus KINERET® (anakinra) blocks the biologic activity of IL-1 by competitively inhibiting IL-1 binding to the IL-1 type I receptor (IL-1RI) and consequently reducing the pro-inflammatory effects of IL-1.

The treatment with these biologic DMARDs relieves symptoms, inhibits the progression of structural damage, and improves physical function in patients with moderate to severely active RA. The three marketed TNF-α blocking agents have similar efficacy when combined with MTX, a widely used DMARD, in the treatment of patients with RA. While providing significant efficacy and a good overall safety profile in the short and medium term in many patients with RA, these biologic treatments may create serious problems and long-term side effects, such as on the liver, and still need to be evaluated. There has been a disturbing association between the use of both of ENBREL® (etanercept) or REMICADE® (infliximab) and the development of lymphoma. As described above, several reports have shown that patients treated with ENBREL® (etanercept) or REMICADE® (infliximab) worsen their congestive heart failure and develop serious infection and sepsis, and increase exacerbations of Multiple Sclerosis and other central nervous system problems.

### Urological Chronic Pelvic Pain Syndrome

Interstitial cystitis or painful bladder syndrome and chronic prostatitis or chronic pelvic pain syndrome were recently renamed as urological chronic pelvic pain syndromes (UCPPS). UCPPS is a chronic disease characterized by a combination of uncomfortable bladder pressure, bladder pain and/or pelvic pain, which can range from mild burning or discomfort to severe pain. It affects approximately 1 million Americans. While it can affect children and men, most of those affected are women. UCPPS can have a long-lasting adverse and significant impact on patient's quality of life. Despite ongoing efforts, neither an effective treatment nor a mechanistic understanding of the pathogenesis of UCPPS exists.

A variety of medications and therapies are available which are based solely on symptom relief. These therapies include: oral medication; bladder instillation (directly into the bladder through a thin, flexible tube inserted through the urethra) using dimethyl Sulfoxide, or instillation of a solution that contains combinations of medications such as the combination of heparin, lidocaine and Sodium bicarbonate); nerve stimulation (transcutaneous electrical nerve stimulation or TENS), using mild electrical pulses to relieve pelvic pain and, in some cases, reduce urinary frequency; bladder distention; and Surgery (bladder augmentation, fulguration, or resection).

Oral medications are the most convenient therapies, which are aimed at relieving pain and/or other symptoms by relaxing and protecting the bladder from irritation by means of pain relievers (Ibuprofen, and nonsteroidal anti-inflammatory drugs); tricyclic antidepressants, such as amitriptyline or imipramine; or antihistamines, like diphenhydramine and loratadine; or pentosan, a semi-synthetically produced heparin-like macromolecular carbohydrate derivative, which chemically and structurally resembles glycosaminoglycans. Among them, pentosan (Elmiron) is the only oral drug approved by the Food and Drug Administration (FDA) specifically for interstitial cystitis. How pentosan works is unknown, but it may line or otherwise restore the inner surface of the bladder, to protect the bladder wall from substances in the urine that could irritate it. It may take several months, however, before patients begin to feel pain relief and up to half a year to experience a decrease in urinary frequency.

Etiology of UCPPS is unknown. However, several hypotheses exist, which include inflammation/autoimmunity, abnormality of cell proliferation, occult infection, genetic, chemical, neurologic, psychological, hormonal, and multifactorial. Among them, proliferation and inflammation probably are the most important. Histologically, it is characterized by thinning or ulceration of the bladder epithelial lining. Cystoscopic abnormalities in the bladder of patients with UCPPS include petechial hemorrhages called “glomerulations” and ulcers that extend into the lamina propria (Hunner's ulcers). The most consistent histologic abnormalities include denudation or thinning of the bladder epithelium to 1-2 cell layers. These findings suggest that UCPPS may be caused by an inhibition of normal bladder epithelial cell proliferation, resulting in a loss of epithelial barrier integrity with subsequent exposure of sensory nerve cells in the bladder wall to urinary constituents. This hypothesis has been supported by the recent identification of a glycosylated frizzled-related peptide inhibitor of cell proliferation from bladder epithelial cells of patients with UCPPS. This antiproliferative factor (APF) significantly inhibits bladder cell proliferation by regulating cell adhesion molecules and growth factor production.

### Multiple Sclerosis

Multiple Sclerosis (MS) is an autoimmune disease diagnosed in 350,000 to 500,000 people in the United States. Multiple areas of inflammation and scarring of the myelin in the brain and spinal cord signify the disease. Patients with MS exhibit varying degrees of neurological impairment depending on the location and extent of the scarring of the myelin. Common symptoms of MS include fatigue, weakness, spasticity, balance problems, bladder and bowel problems, numbness, vision loss, tremors, and depression. Current treatment of MS only alleviates symptoms or delays the progression of disability, and several new treatments for MS including stem cell transplantation and gene therapy are a conservatory. While anti-TNF antibodies have shown protective effects in experimental autoimmune encephalomyelitis (EAE), they aggravate the disease in MS patients, suggesting that inhibition of TNF-α alone is not sufficient.

### Neurodegenerative Disorders

Alzheimer's disease (AD) and Parkinson's disease (PD) are the two most common neurodegenerative disorders. AD is a brain disorder. It seriously affects a person's ability to carry out daily activities. It involves the parts of the brain that control thought, memory, and language. About 4 million Americans, usually after age 60, are estimated to suffer from AD.

PD is a progressive disorder of the central nervous system affecting over 1.5 million people in the United States. Clinically, the disease is characterized by a decrease in spontaneous movements, gait difficulty, postural instability, rigidity, and tremor. PD is caused by the degeneration of the pigmented neurons in the Substantia nigra of the brain, resulting in decreased dopamine availability. The causes of these neurodegenerative disorders are unknown, and there is currently no cure for the disease.

Thus, novel approaches for the treatment of the above and other inflammatory-related diseases are needed. Although the mechanisms by which inflammatory-related diseases are caused remain unclear and often vary from each other, dysfunction of the immune system caused by deregulation of cytokines has been demonstrated to play an important role in the initiation and progression of inflammation.

Cytokines can be generally classified into 3 types: proinflammatory (IL-1α, β, IL-2, IL-3, IL-6, IL-7, IL-9, IL-12, IL-17, IL-18, IL-23, TNF-α, LT, LIF. Oncostatin, and IFNc1α, β, γ); anti-inflammatory (IL-4, IL-10, IL-11, W-13 and TGFB); and chemokines (IL-8, Groα, MIP-1, MCP-1, ENA-78, and RANTES).

In many inflammatory conditions, pro-inflammatory cytokines, especially TNF-α. IL-1β, and IL-6, as well as anti-inflammatory cytokine IL-10 appear to play an important role in the pathogenesis of various inflammatory-related diseases and therefore may serve as potential therapeutic targets. For example, elevated levels of some pro-inflammatory cytokines (TNF-α, IFNγ, IL-1, IL-2, IL-6 and IL-12) and chemokines (IL-8, MCP-1 and RANTES) have been observed in several inflammatory-related diseases such as CD, psoriasis, RA, Grave's disease and Hashimoto's thyroiditis, which parallels an increase in soluble TNF receptors, IL-1 receptor antagonists and the anti-inflammatory cytokine IL-10. IL-10 has been shown to suppress elevated pro-inflammatory cytokine production both in vitro in LPMC cultures and in vivo in patients. Positive response of CD patients treated with IL-10 demonstrates that there might also be an imbalance between the production of pro-inflammatory and anti-inflammatory cytokines in CD.

In Summary, the approach of treating inflammatory-related diseases has undergone an evolutionary change in recent years in part as a consequence of growing concerns of the severity of these diseases and in part due to considerable progress in the understanding of the important role of cytokines in their immuno-pathogenesis. The majority of the efforts have been focused on targeting TNF-α and IL-1, and several products (TNF-α inhibitors: infliximab, a monoclonal anti-TNF-α antibody; and etanercept, the p75 TNF-α receptor) are currently marketed or in clinical trials for the treatment of RA, psoriasis, and IBD as mentioned above. Several other drug candidates or strategies targeting IL-1, IL-6, IL17, IL23 or IL-10 are developed or under development. These biological treatments provide significant efficacy in the short and medium term in many patients with RA or other autoimmune/inflammatory diseases. Although these drugs are well tolerated and have a good overall safety profile, active pharmaco-vigilance is needed. Based on its mechanism of action, and previous notifications of a wide variety of adverse effects, long-term risks of side effects including hematological, infectious, neurological, oncological and immunological effects need to be examined.

Strategies for targeting a single pro-inflammatory cytokine as an anti-inflammatory therapy ignore a very important fact, which is that inflammatory-related diseases involve a sophisticated cytokine network “system.” For example, chemokines, a family of immune molecules related to IL-8 contains approximately 50 ligands and 20 receptors, often acting with redundancy, thus making a selection of appropriate specific antagonists not only difficult but lacking in long-term efficacy. Also, currently marketed products or products under development are mainly protein-based agents, which are expensive to produce and inconvenient to administer (i.e., infusion). Therefore, as functioning of the immune system is finely balanced by the activities of pro-inflammatory and anti-inflammatory mediators or cytokines, modulation of multiple pro/anti-inflammatory cytokines instead of blocking only one particular pro-inflammatory cytokine by Small molecules should not only achieve better therapeutic efficacy with less side effects, but will also have the many advantages of small molecule drugs.

Based on this concept, we previously examined several types of small molecules to test their ability in the regulation of multiple cytokines and explored their potential clinical applications for the treatment of a variety of inflammatory-related diseases.

Meisoindigo is an indirubin derivative that has been used for the treatment of chronic myeloid leukemia (CML) in China with minor side effects. We demonstrated that Meisoindigo and its derivatives are active against solid tumors through their ability to inhibit cyclin-dependent kinases, induce cell differentiation and promote apoptosis. In the current invention, we show novel therapeutic activities of this class of molecules in the treatment of various inflammatory-related diseases including inflammatory bowel diseases and psoriasis in rodents as well as in humans. We demonstrate that this type of agent inhibits the secretion and expression of multiple pro-inflammatory cytokines including IL-1β, IL-6 and TNF-α in cell lines, and promotes production of anti-inflammatory cytokine IL-10. In recent clinical trial, Meisoindigo has been proven very effective against ulcerative colitis while no significant side effects were observed.

Meisoindigo has been shown to be effective in preventing cancer, treating cancer, and treating inflammatory-related diseases associated with pro-inflammatory cytokine expression, reduced expression of anti-inflammatory cytokines, or both.

In certain aspects, the crystalline forms of meisoindigo disclosed herein are stable under conditions of lighting, high temperature and high humidity. The crystalline forms of meisoindigo disclosed herein are also stable under conditions of grinding, pressure and heating, which meets the production, transportation and storage requirements of drug products.

In other aspects, the crystalline forms of meisoindigo disclosed herein are more excellent compared to other crystal forms in view of, for example, purity, handleability (lower hygroscopicity), fluidity, grindability, and/or quality control, and are useful as crystals appropriate for pharmaceutical formulation. These crystalline forms demonstrate excellent stability even in contact with heat, light, oxygen, humidity, or other molecules. Furthermore, the crystal forms of the present invention are excellent in filtration performance, drying characteristics, and fluidity, and can be produced in an industrially advantageous manner.

Moreover, the crystal forms of the present invention, in which the amount of residual solvent is below the reference value described in the Guideline for Residual Solvents in ICH (International Conference on Harmonisation of Technical Requirements for Registration of Pharmaceuticals for Human Use) guidelines, is safe as a medicament. Furthermore, the disclosed crystal forms have a relatively small electric charge amount and are easy to handle in the production and packing of medicaments.

In yet other aspects, the crystal forms of the present invention have one or more beneficial properties, in particular the following advantages: improved stability, improved solubility and dissolution rates in water or in aqueous system(s), improved thermodynamic stability, and/or improved storage stability.

In one aspect, the preparation processes disclosed herein are stable, repeatable and controllable, which makes them suitable for industrial production.

### Preparation of Crystal Form I

### Equipment and Starting Materials

Reaction Vessel: the manufacturing is carried out in a 12 Liter round bottom flask with ports for a thermowell, a reflux condenser and charging reagents and solvents.

Filtration Flask: a 20 Liter Glass Filtration Flask with a side arm to connect to the vacuum.

Tabletop Filter Funnel: a high-density polyethylene funnel measuring 10.25″ inner diameter and 8″ over all height and containing a fixed porous filter plate and fitted with a 12.7 mm vacuum connector.

Starting materials: N-Methylisatin and Oxindole.

### Main Process Steps

The manufacturing process for the production of the bulk N-methylisoindigo (Natura-alpha, NAT) involves the following main steps.

1. Into a 12 L, 3-necked round bottom flask set on a heating mantle and set with an overhead agitator is charged 3.75 L of glacial acetic acid followed by 0.75 kg (4.65 mol) of N-methylisatin and 0.63 kg (4.73 mol) of oxindole and 30 ml of concentrated HCl.

2. The reaction mixture is heated to 85 to 95° C. for 2 to 3 hours.

3. The reaction mixture is allowed to cool to 25 to 30° C. and the precipitated product is filtered by suction.

4. The filter cake is washed with 0.75 L and 0.38 L of glacial acetic acid and sucked to dryness.

5. The filter cake is redissolved in 4.5 L of refluxing glacial acetic acid and the Recrystallized product is filtered by suction, washed with 0.75 L and 0.38 L portions of glacial acetic acid and dried at 50° C. under vacuum.

Natura-alpha (NAT) is prepared by condensing N-methylisatin with oxindole in acetic acid using catalytic concentrated HCl as follows.

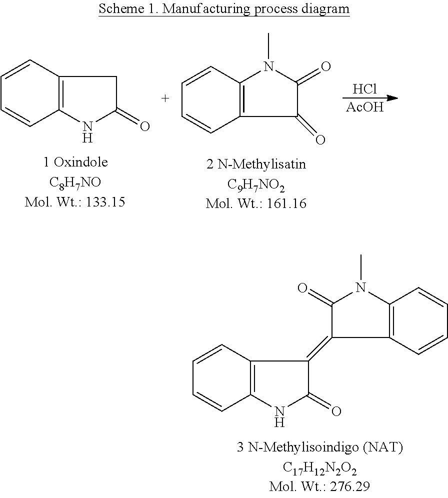

Herein, the inventors also disclose a N-methylisoindigo crystalline composition.

N-methylisoindigo is a dark-red crystalline powder. The melting point of N methylisoindigo is between 235-237° C. N-methylisoindigo is sparingly soluble in acetone, chloroform, and ethanol. N-methylisoindigo is slightly soluble in water.

The structural elucidation and confirmation of N-methylisoindigo is carried out on the primary standard, batch SR-I 205a. The solid state of N-methylisoindigo is presented below (in the “Other Characteristics” Section).

### Elucidation of Structure and Other Characteristics

Elucidation of structure: the structural elucidation and structure of the compound N-methylisoindigo are provided. The analytical techniques used for the structural elucidation are Infrared spectrophotometry, Nuclear magnetic resonance spectrometry, and Mass spectrometry.

### Infrared Spectrum

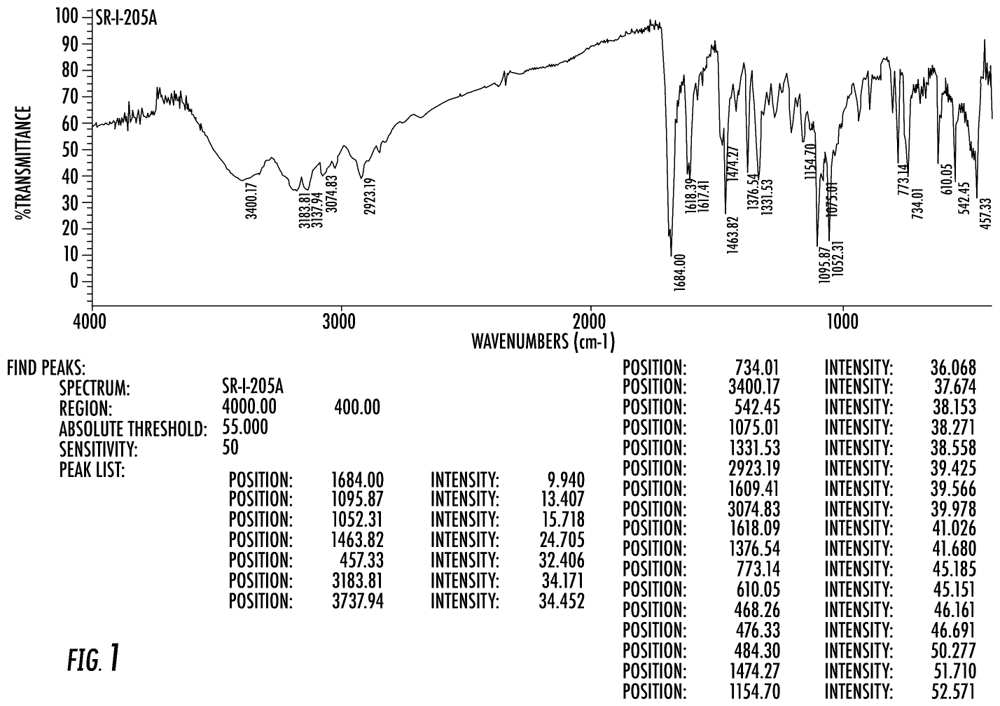

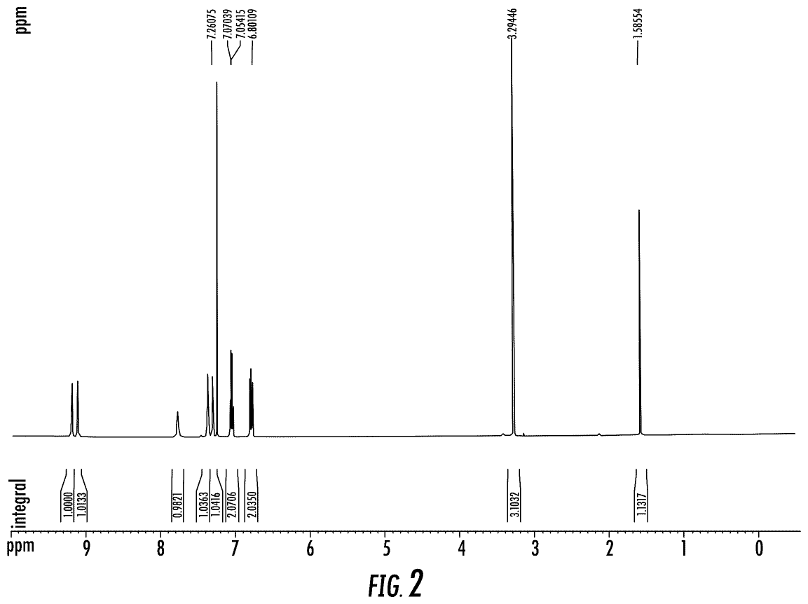

Infrared spectrum of the compound was obtained on the pellet of a 1% mixture of the compound in anhydrous KBr. IR absorptions at 1685 and 1695 cm⁻¹ were characteristic of the carbonyl absorptions of the expected product. The results are provided hereafter in FIG. 1.

### Nuclear Magnetic Resonance Spectrum

Nuclear Magnetic Resonance spectra were recorded by NuMega Resonance Labs, Inc. (San Diego, CA). The spectra were obtained in deuterated chloroform solution using the residual proton resonance at δ 7.27 ppm as the reference peak; proton resonances at δ 3.29 (s, 3H), 6.79 (d, J=8 Hz, 1H), 6.81 (d, J=8 Hz, 1H), 7.06 (dd, J=8, 8 Hz, 1H), 7.08 (dd, J=8, 8 Hz, 1H), 7.31 (dd, J=8,8 Hz, 1H), 7.39 (dd, J=8, 8 Hz, 1H), 7.85 (bs, 1H), 9.12 (d, J=8 Hz, 1H), and 9.19 (d, J=8 Hz, 1H) were consistent with the structure of the expected product. The results are provided hereafter in FIG. 2.

### Mass Spectrometry

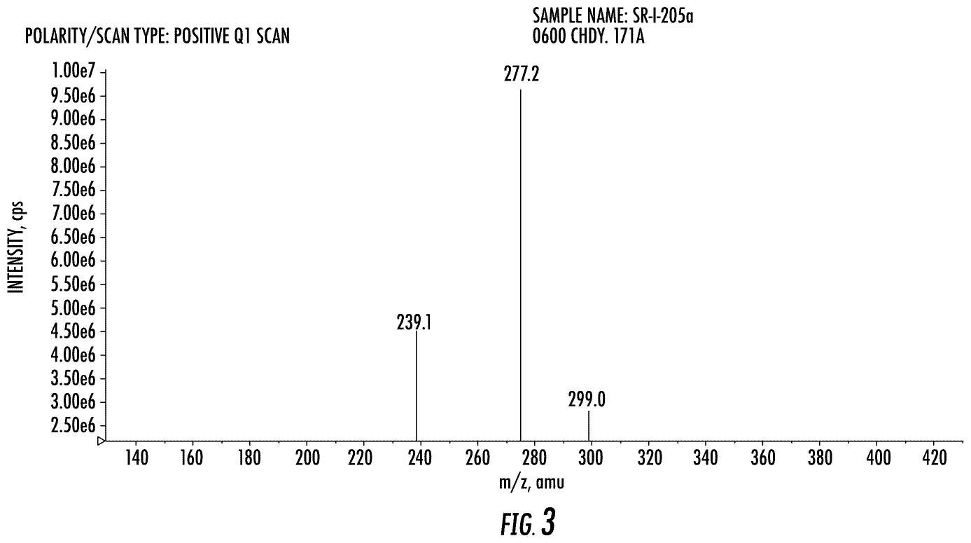

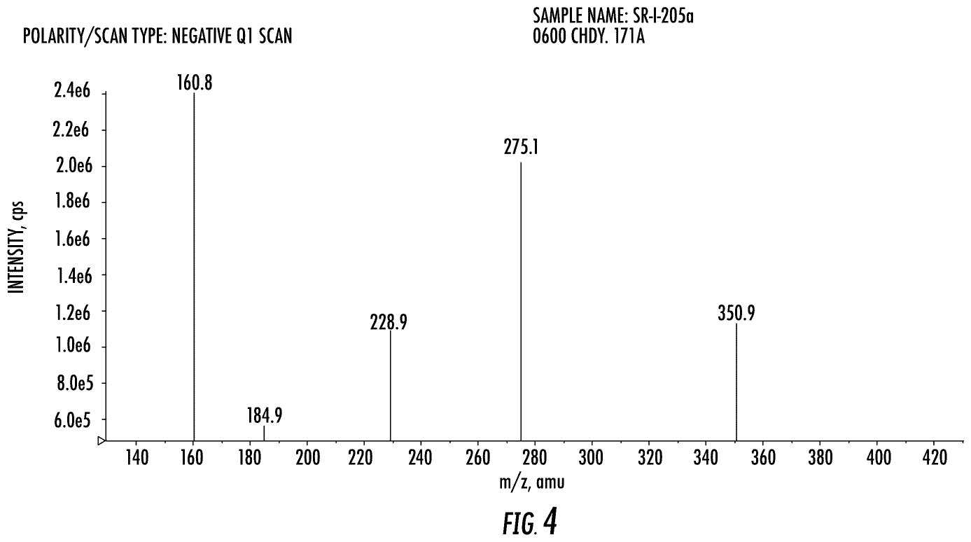

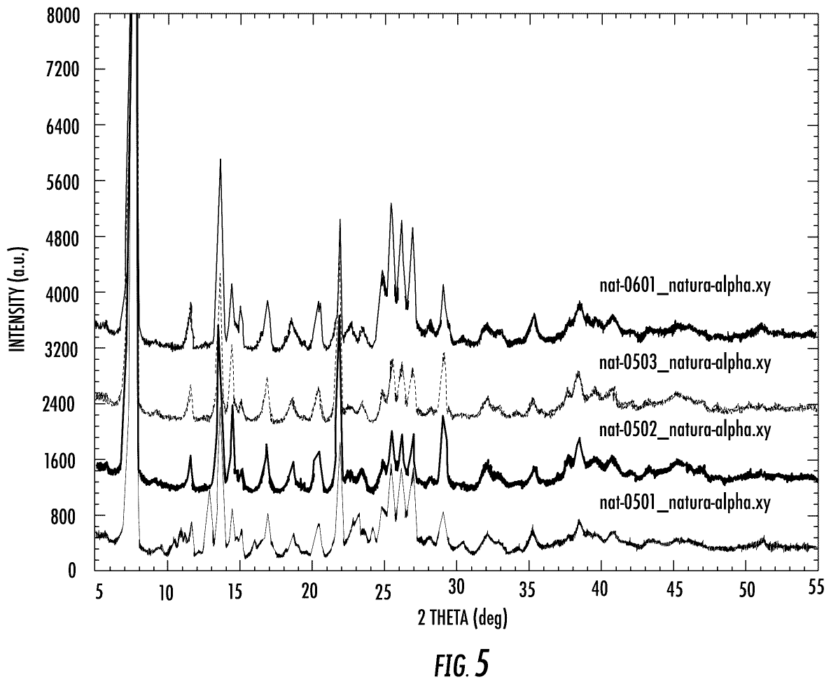

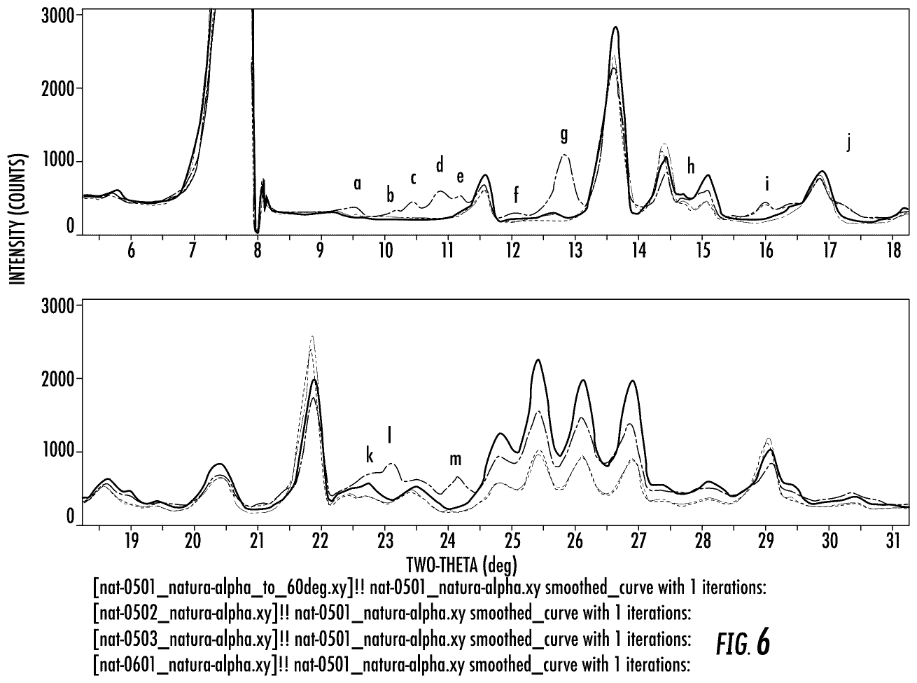

The mass spectra were also recorded by NuMega Resonance Labs, Inc. Ionization were achieved by using Elecrospray ionization method. The positive ion at 277 mass unit in the positive Q1 scan (FIG. 3) and the negative ion at 275 mass unit in the negative Q1 scan (FIG. 4) were characteristic of the M+H and M−H ions respectively of the expected product.

### Other Characteristics

### Solid state

### Melting Point

The melting point was obtained using a capillary tube-type melting point apparatus. The capillaries were 0.8-1.1 mm O.D., 90 mm long, sealed at one end. The melting temperature was measured with a calibrated electronic thermometer using a K-type thermocouple. The melting range should be 235° C.-237° C., with a variation <3° C.

TABLE 1
Melting point of N-methylisoindigo in crystal form (Form 1)

| Batch number | NAT-0501 | NAT-0502 | NAT-0503 | NAT-0601 |
|---|---|---|---|---|
| Melting Point (° C.) | 236.0-236.6 | 235.8-236.3 | 235.4-235.9 | 235.2-236.5 |

The data in Table 1 demonstrate that the batches are comparable between them.

Polymorphism study

Screening of polymorphism

X-ray diffraction patterns were evaluated using four batches of drug substance, NAT-0501, NAT-0502, NAT-0503 and NAT-0601.

The raw x-ray diffraction patterns for the four samples are shown in FIG. 5, with artificial y-axis offsets and 100% peak cutoff (at 2θ=7.71°) for clarity. It is apparent from these four x-ray diffraction profiles that:

1. All four samples from the same manufacturing procedures include the same crystal form of the compound with the one peak at 2θ=7.71° dominates (˜40,000 counts versus 2,000 for the next-highest peak).

2. Samples NAT-0502 and NAT-0503 are nearly identical in crystalline ingredients.

3. Sample NAT-0601 is slightly more crystalline than the others, so that weaker peaks are more apparent, such as at the shoulders of the 17, 18 and 29° peaks.

4. Sample NAT-0501 contains extra peaks, meaning an extra component is in the powder, in a small amount (e.g., several multiple, but minor peaks near 11, 16 and 23°). A possible explanation for this may be that Lot #0501 was reworked. When lot #NAT-0501 was prepared, there was no recrystallization step and the purity was found to be less than 98%. A recrystallization step was added and the product obtained met the specification.

FIG. 6 shows a detailed view of the peak selection analysis in program Jade, and the list of found 2θ values for all four samples are compiled in Table 2.

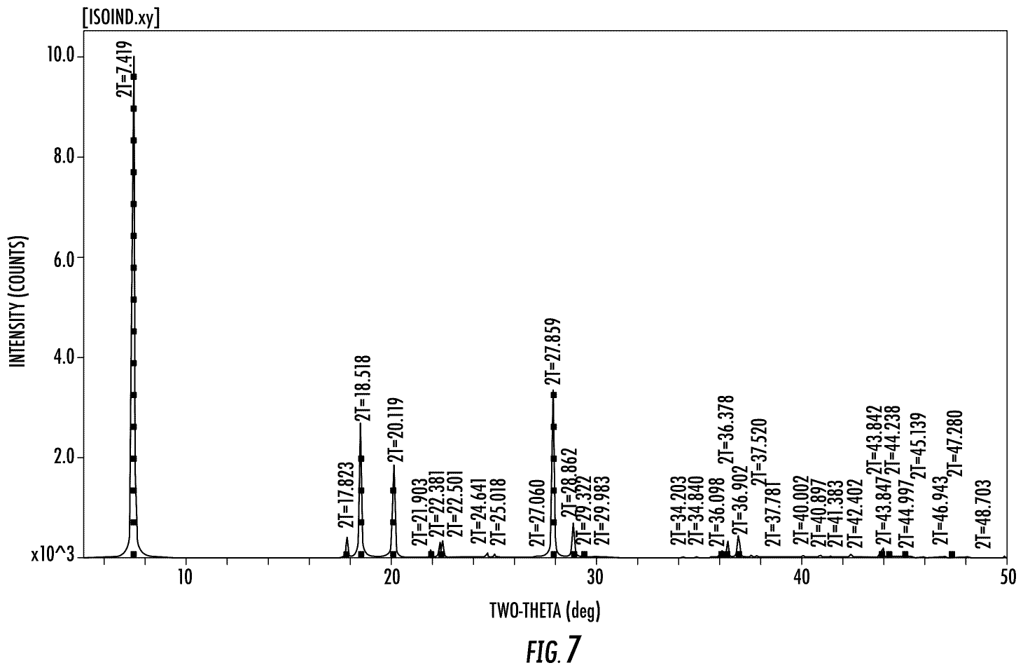

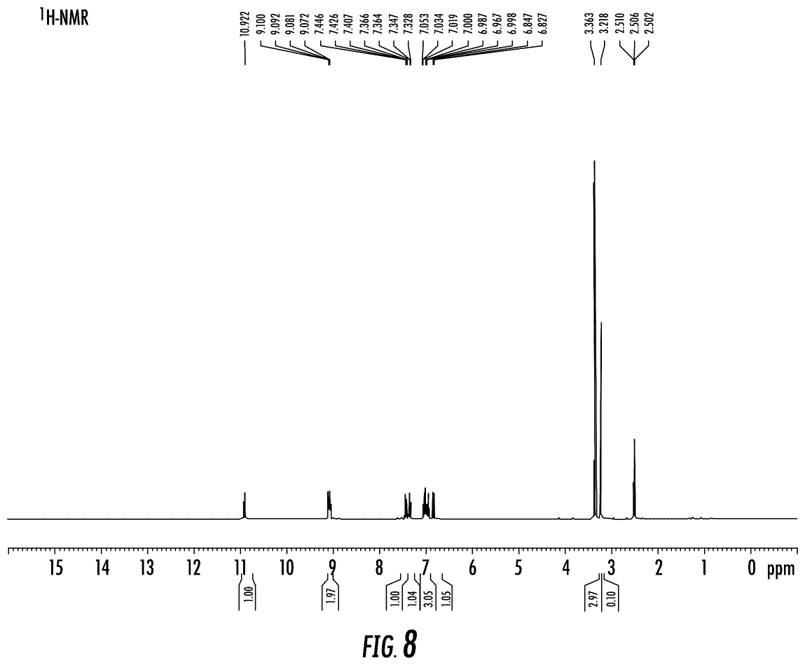

The search/match of characteristic peaks in the powder x-ray diffraction patterns of the samples did not yield any known phases in the powder x-ray databases (PDF 2006 and ICSD 2007). Thus, a determination of the phase could not be made. However, as noted from the appearance of the powder pattern of isoindigo (obtained from the Cambridge Structure Database), the patterns of the samples are consistent with a planar extended molecule of the approximate size and orientation (in the crystal unit cell) as isoindigo, which also has one main peak neat 7.7° and much weaker, but “finger like” region at 13<2θ<30°), as in FIG. 7.

The data were collected on a Philips XPert system with graphite monochromatized Cu Kα x-radiation (λ=1.5418 Å) and a scan step size and speed of 0.02 deg and 0.01 deg sec⁻¹, respectively. The XY plots were analyzed with use of programs WinplotR and Jade.

TABLE 2
List of 2θ values for polymorphism study samples. Observed 2θ angles
(deg) and intensities (counts) for peaks picked in JADE for samples 0501, 0502,
0503, 0601. The average particle size enclosed in parentheses is based upon
the FWHH values of the 3 largest and most isolated reflections. The letters
(a through m) identifying the additional peaks in sample 0501 are also shown on FIG. 6.

| 501 | (28 nm) | | 502 | (28 nm) | 503 | (27 nm) | 601 | (26 nm) |
|---|---|---|---|---|---|---|---|---|
| 2Theta | Intensity | | 2Theta | Intensity | 2Theta | Intensity | 2Theta | Intensity |
| 5.71 | 410 | | 5.77 | 78 | 5.71 | 503 | 5.77 | 566 |
| 6.09 | 282 | | | | | | | |
| 7.71 | 27773 | * | 7.71 | 45547 | 7.69 | 39269 | 7.73 | 35260 |
| 8.29 | 79 | | 8.49 | 134 | 8.39 | 144 | | |
| 9.33 | 99 | | 9.21 | 148 | 9.21 | 143 | 9.19 | 144 |
| 9.53 | 120 | a | 9.51 | 95 | | | | |
| 10.15 | 93 | b | | | | | | |
| 10.43 | 217 | c | | | | | | |
| 10.89 | 369 | d | 10.67 | 48 | | | | |
| 11.19 | 301 | e | | | | | | |
| 11.55 | 455 | | 11.55 | 463 | 11.55 | 451 | 11.57 | 647 |
| 12.11 | 60 | f | 11.93 | 36 | 11.87 | 33 | 12.01 | 17 |
| 12.81 | 868 | g | 12.59 | 70 | 12.67 | 67 | 12.61 | 96 |
| 13.61 | 2063 | * | 13.61 | 2294 | 13.59 | 2073 | 13.63 | 2653 |
| 14.41 | 633 | | 14.39 | 1073 | 14.37 | 968 | 14.41 | 879 |
| 14.71 | 280 | h | 14.71 | 295 | 14.67 | 282 | 14.69 | 365 |
| 15.05 | 387 | | 15.05 | 299 | 15.07 | 294 | 15.07 | 643 |
| 15.55 | 35 | | 15.41 | 52 | | | 15.45 | 48 |
| 16.01 | 223 | i | | | | | | |
| 16.41 | 202 | | | | | | 16.49 | 237 |
| 16.85 | 543 | | 16.83 | 657 | 16.83 | 587 | 16.87 | 676 |
| 17.21 | 205 | j | | | | | | |
| 18.21 | 89 | | 18.21 | 128 | 18.17 | 110 | 18.23 | 174 |
| 18.61 | 315 | | 18.57 | 354 | 18.59 | 327 | 18.65 | 415 |
| 18.97 | 170 | | 19.01 | 115 | 18.99 | 113 | 19.01 | 216 |
| 19.43 | 70 | | 19.41 | 91 | 19.35 | 86 | 19.43 | 109 |
| 20.43 | 461 | | 20.43 | 513 | 20.41 | 476 | 20.43 | 616 |
| 21.45 | 164 | | | | | | | |
| 21.89 | 1395 | * | 21.87 | 2406 | 21.85 | 2218 | 21.89 | 1770 |
| 22.35 | 126 | | 22.33 | 205 | 22.43 | 235 | 22.51 | 289 |
| 22.81 | 417 | k | 22.71 | 225 | 22.75 | 230 | 22.73 | 342 |
| 23.11 | 539 | l | | | | | | |
| 23.49 | 332 | | 23.43 | 292 | 23.41 | 275 | 23.49 | 290 |
| 24.15 | 343 | m | | | | | | |
| 24.81 | 656 | | 24.83 | 387 | 24.81 | 392 | 24.85 | 1019 |
| 25.43 | 1258 | | 25.47 | 764 | 25.45 | 832 | 25.45 | 2001 |
| 26.13 | 1176 | | 26.13 | 733 | 26.13 | 739 | 26.15 | 1733 |
| 26.89 | 1070 | | 26.93 | 706 | 26.91 | 712 | 26.91 | 1723 |
| 27.29 | 135 | | 27.33 | 136 | 27.37 | 129 | 27.31 | 305 |
| 27.53 | 112 | | | | | | | |
| 28.17 | 96 | | 28.11 | 163 | 28.11 | 149 | 28.11 | 339 |
| 29.09 | 476 | | 29.07 | 976 | 29.05 | 891 | 29.09 | 768 |
| 29.39 | 135 | | 29.39 | 145 | 29.39 | 130 | 29.39 | 276 |
| 30.39 | 128 | | 30.43 | 58 | 30.45 | 45 | 30.43 | 94 |
| 31.77 | 167 | | | | 31.77 | 172 | 31.69 | 150 |
| 32.15 | 276 | | 32.07 | 311 | 32.07 | 278 | 32.11 | 294 |
| 32.37 | 213 | | | | | | | |
| 32.85 | 169 | | 32.85 | 168 | 32.81 | 152 | 32.89 | 206 |
| 33.15 | 138 | | | | | | 33.05 | 188 |
| 35.25 | 242 | | 35.31 | 233 | 35.27 | 222 | 35.27 | 359 |
| 36.87 | 110 | | | | | | | |
| 37.73 | 221 | | 37.69 | 344 | 37.67 | 344 | 37.77 | 292 |
| 38.49 | 411 | | 38.47 | 605 | 38.39 | 556 | 38.55 | 503 |
| 39.03 | 245 | | 39.13 | 257 | 39.15 | 247 | 39.07 | 292 |
| 39.55 | 217 | | 39.55 | 316 | 39.55 | 307 | 39.59 | 307 |
| 40.53 | 184 | | 40.33 | 243 | 40.27 | 227 | 40.49 | 256 |
| 40.75 | 217 | | 40.59 | 308 | 40.83 | 298 | 40.79 | 334 |
| 40.99 | 170 | | 40.81 | 314 | 41.11 | 158 | 40.99 | 269 |
| 41.57 | 69 | | 41.89 | 85 | 41.47 | 66 | | |
| 41.99 | 26 | | 42.09 | 100 | 42.11 | 98 | 42.11 | 106 |

The terms “about” and “substantially as shown in” refer typically to ±0.2° for X-ray powder diffraction (XRPD) peaks and to ±3° C. for differential scanning calorimetry (DSC).

### Particle Size and Distribution

Crystal Form I was analyzed for its particle size and distribution. Five batches were tested using a Malvern Mastersizer 2000 (MIIA14730, Malvern, UK).

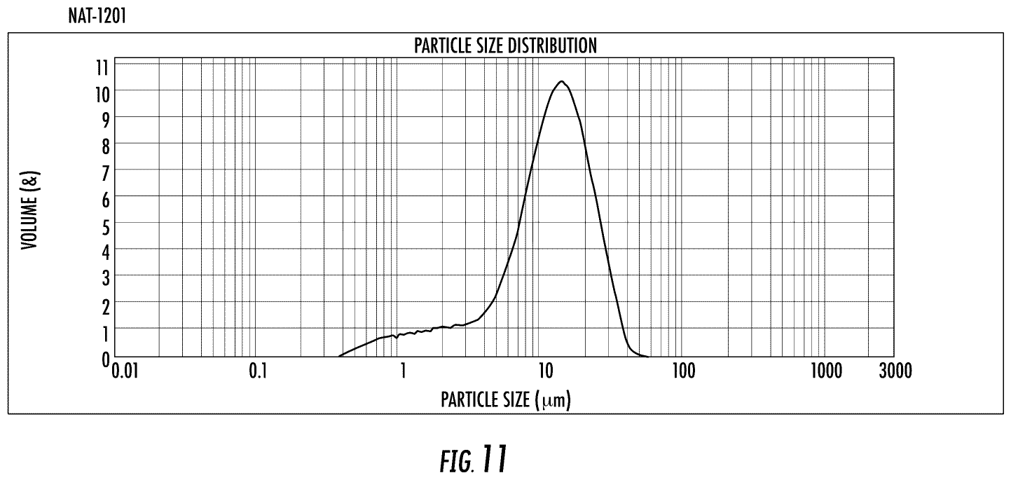

As shown in Table 3, the particle size from five batches of the Crystal Form I was found to be very uniform and distributed evenly. FIG. 11 shows examples of particle distribution of the Crystal Form I of the compound. These physical properties facilitate pharmaceutical processing, such as formulation of various solid and topical formulations as needed without additional processing of the raw active pharmaceutical ingredient (API) material. The physical properties also contribute to equivalent and stable bioavailability between batches.

TABLE 3
Results of particle size determinations from five batches of Crystal Form I of
Meisoindigo. The test was performed using the Malvern Mastersizer 2000 (MIIA14730).
The particle size distribution ratios were calculated as (d90-d10)/d50.

| | | | | | | Particle Size |
|---|---|---|---|---|---|---|
| | Exova | | | | | Distribution |
| Client Sample | Sample # | Parameter | Specification | Result | | Ratio |
| Natura-alpha | 87301 | d10 | Report Results μm | 3.599 | μm | 1.75 |
| NAT-1201 | | d50 | Report Results μm | 13.301 | μm | |
| | | d90 | Report Results μm | 26.897 | μm | |
| Natura-alpha | 87302 | d10 | Report Results μm | 2.506 | μm | 1.86 |
| NAT-0601 | | d50 | Report Results μm | 12.464 | μm | |
| C00105 | | d90 | Report Results μm | 25.635 | μm | |
| Natura-alpha | 87304 | d10 | Report Results μm | 5.035 | μm | 1.62 |
| NAT-1202 | | d50 | Report Results μm | 15.603 | μm | |
| | | d90 | Report Results μm | 30.339 | μm | |
| Natura-alpha | 87305 | d10 | Report Results μm | 3.249 | μm | 1.69 |
| NAT-0801 | | d50 | Report Results μm | 12.268 | μm | |
| C00105 | | d90 | Report Results μm | 23.99 | μm | |
| Natura-alpha | 87306 | d10 | Report Results μm | 5.295 | μm | 1.58 |
| NAT-1203 | | d50 | Report Results μm | 15.674 | μm | |
| C00105 | | d90 | Report Results μm | 30.015 | μm | |

The experimental data indicate that Crystal Form I has a uniform particle size with an ideal distribution (see FIG. 11). This uniform particle size facilitates formulation of Crystal Form I into a pharmaceutical composition. The uniform particle size also indicates the reproducibility of the process for forming Crystal Form I.

### Preparation of Additional Crystal Forms

After exploring different synthesis routes and different crystallization/recrystallization conditions produce different forms of crystalline of N-methylisoindigo (Natura-alpha), the inventors identified the following additional conditions of synthesis and crystallization/recrystallization. The resulting products were examined using X-ray powder diffraction (XRD).

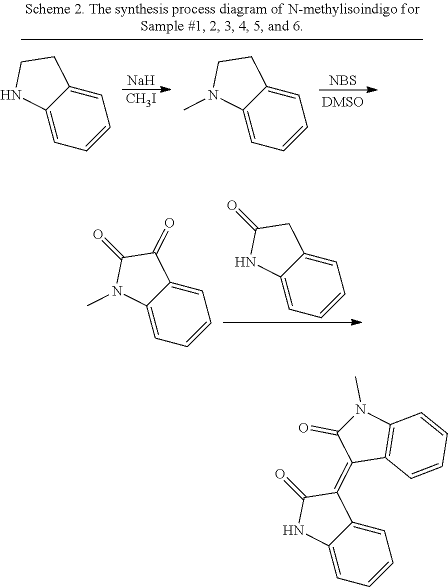

Indole was reacted with methyl iodide using NaH as a catalyzer to produce methyl indole which was oxidized to become 1-methyl indigo under N-bromosuccinimide (NBS) and dimethyl sulfoxide (DMSO). 1-Methyl indigo was then reacted with 2-oxindole under acidified condition to produce N-methylisoindigo. The compound was then purified by column chromatograph, recrystallized under the different conditions described below, and the structure characterized using ¹H-NMR, ¹³C-NMR, and LC-MS. Representative spectra are shown in FIGS. 8-9.

Sample #1: recrystallization in methanol/water: Three hundred (300) mg of newly synthesized N-methylisoindigo was heated to 80° C. to which 80 ml of methanol was added, and heated under reflux until it became clear. Two hundred (200) ml of water was then slowly added to the solution with heat reflux. The solution became cloudy again and was heated under reflux continuously until it became clear again, and then filtered when it was still hot. The solution stood and cooled down to room temperature. The compound gradually precipitated and was then filtered and dried.

Sample #2: recrystallization in methanol: Three hundred (300) mg of newly synthesized N-methylisoindigo was heated to 80° C. to which 80 ml of methanol was added, and heated with reflux until it became clear. The solution was then filtered when it was still hot, stood and cooled down to room temperature. The compound gradually precipitated and was then filtered and dried.

Sample #3: recrystallization in ethyl acetate and ligroin: One hundred (100) mg of newly synthesized N-methylisoindigo was heated to 80° C. to which 40 ml of ethyl acetate was added under heat reflux until it became clear. One hundred twenty (120) ml of ligroin was then slowly added to the solution. The solution became cloudy and was then heated under reflux until it became clear again. The solution was filtered when it was still hot, stood and cooled down to room temperature. The compound gradually precipitated and was then filtered and dried.

Sample #4: recrystallization in ethyl acetate: One hundred (100) mg of newly synthesized N-methylisoindigo was heated to 80° C. to which 30 ml of ethyl acetate was added under heat reflux until it became clear. The solution was then filtered when it was still hot, stood and cooled down to room temperature. The compound gradually precipitated and was then filtered and dried.

Sample #5: recrystallization in ethyl alcohol and water: One hundred (100) mg of newly synthesized N-methylisoindigo was heated to 80° C. to which 30 ml of ethyl alcohol was added under heat reflux until it became clear. Sixty (60) ml of distilled water was then added to the solution. The solution became cloudy temperately and then clear again. The solution was filtered when it was still hot, stood and cooled down to room temperature. The compound gradually precipitated and was then filtered and dried.

Sample #6: recrystallization in ethyl alcohol: One hundred (100) mg of newly synthesized N-methylisoindigo was heated to 80° C. to which 30 ml of ethyl alcohol was added under heat reflux until it became clear. The solution was filtered when it was still hot, stood and cooled down to room temperature. The compound gradually precipitated and was then filtered and dried.

Samples 1 to 6 were analyzed for purity, and each sample was found to be >98.00% pure.

Some physical properties of the sample #1 to #6 are shown in Table 4. The appearance of these crystal forms is different: sample #1 and #5 are powder like; sample #2 and #4 are arborescent whereas sample #3 and #6 are acicular. Both arborescent and acicular are known to have a poor flow rate and are, thus, not ideal for pharmaceutical formulations. Importantly, all these crystalline forms showed lower milting points (i.e., 225.4° to 229.5° C. as compared with 235.2° to 236.6° C. disclosed above for Crystal Form I) demonstrating these crystal forms are different from Crystal Form I.

TABLE 4
Physical properties of samples #1 to #6

| | | Crystalline | |
|---|---|---|---|
| Sample # | Color | Property | Melt point |
| 1 | Brown | Powder-like | 226.4° C.~227.2° C. |
| 2 | Brown | Arborescent | 226.7° C.~227.6° C. |
| 3 | Henna Mahogany | Acicular | 228.3° C.~228.9° C. |
| 4 | Henna Mahogany | Arborescent | 227.8° C.~229.3° C. |
| 5 | Henna Mahogany | Powder-like | 228.0° C.~229.5° C. |
| 6 | Henna Mahogany | Acicular | 225.4° C.~228.3° C. |

Structure characterization of the newly synthesized N-methylisoindigo was done by ¹H NMR, ¹³C-NMR and LC-MS, and representative spectra are shown in FIGS. 8 and 9.

FIGS. 10A-10G show the X-ray spectrum (PXRD) of the 6 crystalline samples. None of them have repeated each other. PXRD suggests that sample 1-6 were mixtures of multiple crystalline forms.

In comparison with the other samples, the XRD spectra of sample 1 and sample 5 displayed many narrow and sharp peaks, indicating a relatively good crystalline state of meisoindigo produced. However, relative wide peaks occurred at larger θ, implying non-crystalline forms were also present. Relatively, sample 1 exhibited a better crystalline state of meisoindigo than that of sample 5 as it produced more sharp and strong peaks with a shorter melting range than did sample 5.

Sample 2 and sample 4 showed fewer narrow peaks and fewer high peaks, indicating the crystalline forms not only had larger particle sizes but were mixtures of crystal types.

Sample 3 and 6 showed peaks that were not only narrow but relatively impure compared to the other samples. Many peaks from these samples overlapped each other, indicating a typical mixture of crystal types of meisoindigo. The number of crystal types in Sample 3 and 6 were much more than that of the other 4 samples. This indicates that many crystalline forms were quickly produced under the given re-crystalline conditions, and the crystalline forms might contain the solvent. These results suggest that the re-crystalline conditions for Sample 3 and 6 were not suitable to yield a uniform crystalline powder.

Since N-methylisoindigo chemically is classified under the “conjugated aromatic lactams,” hydrogen bonds (H-bonds) and x-stack structures easily form between molecules. Therefore, it is difficult to dissolve these compounds in a solvent, and it is easy to form crystal and non-typical crystal forms that may contain solvent during crystallization. The crystal forms of samples 1 to 6 contain particles that are not uniform and have a poor flow rate. This makes it more difficult to formulate the crystal forms of samples 1 to 6 without additional processing procedures. In contrast, Crystal Form I is unique in its properties with uniform particle size, flowability, and reproducibility in its production and isolation.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein. All publications, patents, and patent publications cited are incorporated by reference herein in their entirety for all purposes.

The publications discussed herein are provided solely for their disclosure prior to the filing date of the present application. Nothing herein is to be construed as an admission that the present invention is not entitled to antedate such publication by virtue of prior invention.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

We claim:

1. A solid form of N-methylisoindigo or a solid crystal form of N-methylisoindigo (Crystal Form I) having an X-ray powder diffraction pattern comprising a peak, in terms of 2-theta, at about 7.71°.

2. The solid form or solid crystal form of claim 1 having an X-ray powder diffraction pattern comprising peaks, in terms of 2-theta, at about 7.71°, about 17°, about 18°, and about 29°.

3. The solid form or solid crystal form of claim 1 or 2, having an X-ray powder diffraction pattern substantially as shown in FIG. 5.

4. The solid form or solid crystal form of any one of claims 1 to 3, having an infrared spectrum of N-methylisoindigo substantially as shown in FIG. 1.

5. The solid form or solid crystal form of any one of claims 1 to 4, having an NMR spectrum of N-methylisoindigo substantially as shown in FIG. 2.

6. The solid form or solid crystal form of any one of claims 1 to 5, having a differential scanning calorimetry (DSC) thermogram comprising an endothermic peak at about 235° C. to 237° C.

7. The solid form or solid crystal form of any one of claims 1 to 6, having a differential scanning calorimetry (DSC) thermogram substantially as shown in Table 1.

8. The solid form or solid crystal form of any one of claims 1 to 7, having a particle size distribution with an average particle size d50 below 25 μm.

9. The solid form or solid crystal form of claim 8, having a particle size distribution ratio (d90-d10)/d50 of less than 2.50 and a maximum particle size below 100 μm.

10. The solid form or solid crystal form of claim 8 or 9, wherein the particle size distribution facilitates pharmaceutical processing during formulation and enhances the stability and bioavailability of the solid form or solid crystal form.

11. A pharmaceutical composition comprising the solid form or solid crystal form of any one of claims 1 to 10, and a pharmaceutically acceptable carrier.

12. The pharmaceutical composition of claim 11, wherein the solid form or solid crystal form is present in said composition in an amount of at least about 90% by weight.

13. The solid form or solid crystal form of any one of claims 1 to 10, which is substantially purified.

14. The solid form or solid crystal form of any one of claims 1 to 10, which is crystalline.

15. A process for preparing the solid form or solid crystal form of any one of claims 1 to 10, the process comprising precipitating a crystalline form from a solution comprising an organic solvent.

16. The process of claim 15, wherein the solution comprises glacial acetic acid.

17. The process of claim 16, wherein the solution further comprises N-methylisatin, oxindole, and/or HCl.

18. A solid form or a solid crystal form of N-methylisoindigo prepared by the process of any one of claims 15 to 17 and recrystallized in any other acceptable organic solvent, and preferably in glacial acetic acid.

19. A method of treating cancer, comprising administering the solid form or solid crystal form of any one of claims 1 to 10 or the pharmaceutical composition of any one of claims 11 to 12 to a patient in need thereof.

20. A method of treating an inflammatory-related disease associated with cytokine expression levels, comprising administering the solid form or solid crystal form of any one of claims 1 to 10 or the pharmaceutical composition of any one of claims 11 to 12 to a patient in need thereof.
